(19) United States
(12) Patent Application Publication
(10) Pub. No.: US 2011/0009327 A1
(43) Pub. Date: Jan. 13, 2011

(54) BIOACTIVE NANOCOMPOSITE MATERIAL

(75) Inventors: Robert Graham Hill, Berkshire (GB); Gowsihan Poologasundarampillai, London (GB); Julian R. Jones, Surrey (GB)

Correspondence Address: Pepper Hamilton LLP, 400 Berwyn Park, 899 Cassatt Road, Berwyn, PA, 19312-1183, US

(73) Assignee: IMPERIAL INNOVATIONS LIMITED, London (GB)

(21) Appl. No.: 12/676,644

(22) PCT Filed: Sep. 5, 2008
(86) PCT No.: PCT/GB2008/003008

§ 371 Date: Sep. 25, 2010

(30) Foreign Application Priority Data
Sep. 7, 2007 (GB) 0717516.9

(51) Int. Cl. A61K 38/00; C07K 14/00; A61P 19/04

(52) U.S. Cl. 514/16.7; 530/350; 530/354; 530/356; 977/894; 977/795

(57) ABSTRACT

The present invention relates to a porous inorganic/organic hybrid nanoscale composite comprising an enzymatically biodegradable organic polymer and a sol-gel derived silica network, its production and use as a macroporous scaffold in tissue engineering.

The present invention relates to an inorganic/organic hybrid nanoscale composite, its production and use as a macroporous scaffold in tissue engineering.

As healthcare is improving and life expectancy increases we are outliving our body parts, including our bones. Bone grafting procedures are used to regenerate bone that has been removed or damaged due to disease and trauma. More than 300 000 bone graft operations are performed in Europe each year. Current surgical best practice is to remove healthy bone from the iliac crest (autograft), and place it into the desired location. While effective, this procedure requires additional surgical time (an extra invasive operation) and can produce post-operative pain at the site of bone removal and a long recovery time. The bone is also in limited supply. A more plentiful supply of bone are allografts; bone sourced from bone banks, which distribute bone from cadavers. These bones do not usually have the mechanical strength of autografts and there is a chance of immunorejection and disease transmission. A patient may require lifetime treatment with expensive immunosuppressant drugs that can also yield dangerous side effects. Animal bones (xenograft) can also be used, e.g. freeze dried bovine bone, but mechanical properties are poor and there is still the risk of disease transmission.

Bone grafts are used in: (i) maxillofacial surgery, (ii) in orthopaedics to repair defects created due to trauma, tumours and cysts, and (iii) in dentistry, where they are often used to cure periodontitis (bone loss at the tooth root). Many surgical procedures of the spine, pelvis and extremities require grafts. Bone grafts may also be needed in situations where healing may be difficult due to nicotine use, or the presence of diseases such as diabetes or autoimmune deficiencies.

A regenerative scaffold is particularly important in the elderly and in the young. All tissues in elderly people are slow to heal due to lack of active cells. Therefore a synthetic bone-healing material that is available off the shelf for a surgeon to immediately implant into a bone defect would dramatically improve quality of life of patients across the globe.

One of the most common uses of bone grafts in spine surgery is during spinal fusion, which is a vital operation needed to reduce debilitating pain. One of every 700 newborns has a cleft pallet. Maxillofacial surgery with materials that respond to the physiological environment are vital so that the regenerative site can remodel as the child grows.

Biomaterials can be used in biomedical applications, specifically tissue regeneration and tissue engineering, and can replace bone grafts. Such regenerative bone graft substitutes have the potential to greatly improve healthcare treatments and quality of life of patients. A biologically active (or bioactive) material is one which, when implanted into living tissue, induces formation of an interfacial bond between the material and the surrounding tissue.

Typically strategies for promoting bone regeneration involve use of a scaffold material. A scaffold is a template on which bone can grow in three dimensions (3D), creating a construct of tissue and scaffold. The two main bone regeneration strategies involving use of a scaffold are in situ tissue regeneration and tissue engineering. Commonly, tissue engineering involves growing cells on a scaffold in a bioreactor outside the body and then implanting the scaffold, after which the scaffold should dissolve as the bone remodels into mature bone. In in situ tissue regeneration, a scaffold is implanted directly into the body. In both cases, the implanted scaffold materials must adapt to the physiological environment. An ideal scaffold for bone repair should: 1) act as template for bone growth in three dimensions; 2) be biocompatible (not toxic); 3) form bonds with host bone (a property referred to as “bioactivity”) and stimulate bone growth; 4) dissolve at a controlled rate with non-toxic degradation products; 5) have mechanical properties matching that of the host bone on implantation; and 6) be capable of commercial production and sterilisation for clinical use.

In order to fulfil criterion 1, the scaffold should have a pore network that is interconnected in 3D, with interconnections large enough to allow cell migration, fluid flow (nutrient delivery), and bone to grow in 3D. The minimum interconnect size for bone with a blood supply to grow in is thought to be 100 μm.

Cells require signals to stimulate them to lay down new tissue. The signals are usually provided by growth factors or hormones. In bone tissue engineering, the signal can either be provided by additives to the bioreactor or delivered by the material. For in situ bone regeneration, they must be delivered by the material.

Bioceramics are often used to form scaffolds for use in hard tissue repair. A material that has the potential to fulfil many of the criteria for an ideal scaffold is bioactive glass. The first bioactive glass was discovered by Hench and was termed Bioglass®, which has been used clinically since the mid-1980s as a regenerative bone filling powder under the product names Perioglas® and Novabone®. Bioactive glasses bond to bone because a hydroxycarbonated apatite (HCA) layer forms on their surface on contact with body fluid. HCA is similar in composition to bone mineral and forms a strong bond therewith. Bioactive glasses dissolve safety in the body, releasing critical concentrations of silicon and calcium ions which act to stimulate bone cells at the genetic level, triggering new bone growth even when few active cells are present. This is particularly important for older patients.

Whilst bioactive glasses are suitable for use as regenerative materials, the Bioglass® composition is unsuitable for the production of porous scaffolds. This is because a sintering process must be employed, which requires glasses to be heated above their glass transition temperature in order to initiate localised flow. The Bioglass® composition crystallises immediately above its glass transition temperature and once Bioglass® crystallises, it loses its bioactivity.

There are however two types of bioactive glass; melt-derived and sol-gel derived. By foaming sol-gel derived silica based bioactive glasses, porous scaffolds have been developed (WO02/096391). Cell response studies on such scaffolds have found that primary human osteoblasts lay down mineralized immature bone tissue thereon, without additional signalling species (Jones et al, Biomaterials, 2007, 28, 1653-1663). Bioactive glasses provide signals, in the form of release of silicon and calcium ions, required for these processes to occur.

Sol-gel derived bioactive glass scaffolds can largely fulfil the criteria for an ideal scaffold, apart from their mechanical properties. Bioactive glass scaffolds can be used in sites that will be under compressive loading, but they cannot be successfully used in sites that are under cyclic loading because the bioactive glasses are brittle. Scaffold materials with improved toughness are therefore required.

A strategy that has been employed to improve toughness of scaffold material is the creation of a composite with a biodegradable polymer. There are many candidate biodegradable polymers that have been considered for bone tissue engineering. Biodegradable polymers break down in the body into products that can be safely secreted by the body. Degradation can either be by hydrolysis (chain scission) after water uptake or by enzymatic mechanisms. Biodegradable polymers can be used either alone or in combination with other bioactive inorganic fillers such as hydroxyapatite or bioactive glass.

Composite materials prepared by dispersion of bioactive glass powder within a polymer solution prior to foaming are known (Maquet et al, J. Biomed. Mat. Res., 66A: 335-346, 2003). However, these conventional composites have several problems which have limited their use. The bioactive inorganic material is often covered by the polymer matrix, which isolates it from the body, causing no bioactivity to be observed. The bioactive phase may be exposed once the polymer degrades, however the rate of degradation of commonly used polymers is often initially slow but then rapidly increases. Rapid degradation can lead to the inorganic phase free in the body, but also rapid loss of mechanical properties of the scaffold. The reason for the slow and then rapid degradation is that the polymers are often polyesters which degrade by hydrolysis (chain scission). As the chains are cut, the molecular weight of the polymer drops and at a critical value the polymer will fall apart. This process is accelerated by the acidic degradation products of the polymers.

A potential way of overcoming these problems is the development of inorganic/organic nanocomposite scaffolds, in which inorganic chains with nanometer dimensions are combined with a polymer matrix. Inorganic/organic nanocomposites, are sometimes referred to as hybrids, ormosils or ceramers. Such a material would be a close mimic of bone, which is essentially a natural nanocomposite of hydroxycarbonate apatite and collagen.

A bioactive glass/bioresorbable polymer nanoscale composite can be made by varying the sol-gel process, adding a soluble polymer to the sol before the sol-gel transition takes place. However, most biodegradable polymers are not soluble in aqueous solutions.

A bioactive glass/polymer hybrid scaffold comprising polyvinyl alcohol (PVA) has been developed by modification of the sol-gel foaming process (Pereira, et al. Journal of Materials Science: Materials in Medicine, 2005: 16: 1045-1050). PVA dissolved in water was added to a typical sol used to synthesise bioactive glass comprising 70 mol% SiO₂, 30 mol % CaO (70530C). Hybrids were created containing up to 30 wt % polymer. The scaffolds produced had high porosity, varying between 60-90%, and a macropore diameter up to 500 gm. Compression testing on these foams demonstrated that polymer addition resulted in significantly higher compression strength (˜3 fold increase). Increases were also noted in toughness and strain to failure. However, the ultimate failure strength was low compared to trabecular bone. This is at least partially attributable to the low molecular weight of PVA used (MW of 16,000). Low molecular weight PVA cannot act to effectively toughen the scaffold since the toughness of a thermoplastic is dependant on chain pull-out and disentanglement and is highly dependant on molecular weight. Whilst being too low for significantly enhanced toughness, this molecular weight was necessary because PVA is non-degradable and larger chains will not pass through the kidneys. Moreover, any condensation of silanols with pendant hydroxyl groups occurs extremely slowly, if at al, and therefore these hybrids relied largely on hydrogen bonding between the organic and the inorganic chains. In order to provide stability in aqueous environments, covalent bonding is required between the two phases.

Coupling agents can be used to induce covalent bonds between organic and inorganic phases. Coupling agents have been used in the production of bioactive glass/polycaprolactone (PCL) hybrids (Rhee, et al, Biomaterials 25(7-8): 1167-1175 (2004); Rhee, et al., Biomaterials 23(24): 4915-4921 (2002); Tian, et al., Polymer 37(17): 3983-3987 (1996)). PCL is a polyester that is insoluble in aqueous solutions and has to be functionalised in order for it to be incorporated in the sol. In these studies, hydroxyl groups at either end of polycaprolactone diol were targeted by 3-isocyanatopropyl triethoxysilane (IPTS), resulting in a polymer end capped with a triethoxysilyl group. The end capped PCL can then be hydrolysed and co-condensed with TEOS to yield an interconnected polymer-silica network. In some instances, calcium was incorporated into the sol in the form of calcium nitrate tetrahydrate. Bioactive glass/PCL hybrids with 60 wt % polymer showed promising results, having a Young's modulus and tensile strength of 600 and 200 MPa respectively, which is in the range of cancellous bone. However the mechanical properties are limited by the molecular weight of the polymer, which was just 7000. Porous scaffolds were not produced. Were pores to be introduced into these hybrids, their modulus and strength would be expected to fall.

A silica/hyperbranched aliphatic polyester hybrid has also been synthesised using a commercially available polyester (Boltom™ H20) which has 16 hydroxyl groups at the terminals and the molecular weight of 1747 g mol⁻¹ (Zou et al., Composites Part A: Applied Science and Manufacturing, 36(5): 631-637 (2005). The polymer is pre-treated with succinic anhydride to obtain carboxylic group endcaps. Glycidoxypropyltrimethoxysilane (GPTMS) was then added, which bonds to the carboxylic groups to give the polymer chains Si(OCH)₃ endcaps. The modified polymer was added to a sol of pre-hydrolysed TEOS and a co-condensation reaction followed yielding a silica/polymer network.

The hybrids described above are made with polyesters that have unpredictable degradation rates and make use of materials that are toxic to the human body. Moreover, generally hybrid foams and calcium additions have not been demonstrated. The reason that they do not contain calcium is that the conventional method for introducing calcium to a sol-gel glass is to add calcium nitrate into the sol-gel reaction. As the process temperature is raised to above 600° C., the calcium is incorporated in the glass network and the nitrates are burnt off. High temperatures are not possible when polymers are present, as they would burn off. Alternatively, if a calcium nitrate is incorporated into a sol-gel reaction in a process that does not involve heating sufficient to burn off the nitrate, nitrates may be present in the final hybrid product leading to possible toxicity. Therefore, there is a need for a new means of incorporation of a source of calcium ions which does not require high temperature treatment and avoids potential toxicity of residual nitrate.

The above examples demonstrate that there are complex problems to solve in the development of bioactive inorganic/organic nanocomposite scaffolds for tissue regeneration. There is a need for a biocompatible porous scaffold that can act as template for bone growth in three dimensions, that has the appropriate mechanical properties to allow use for bone regeneration in load bearing sites, that is degradable at a controlled rate, that contains a source of calcium ions to provide bioactivity and stimulate bone growth and that is capable of commercial production and sterilisation for clinical use. It has now been determined that a scaffold fulfilling these criteria can be produced in the form of a nanocomposite material comprising an inorganic phase and an organic polymeric phase, with the correct choice of organic polymer and use of a crosslinker to ensure covalent bonding between the organic and inorganic phases.

Therefore, in a first aspect the present invention provides a porous composite material comprising an organic phase and an inorganic phase, wherein the organic and inorganic phases are integrated and wherein the organic phase comprises an enzymatically biodegradable organic polymer and the inorganic phase comprises a sol-gel derived silica network, wherein covalent bonding is present between the organic phase and the inorganic phase and wherein the composite material comprises a source of calcium and/or strontium ions.

Preferably, the composite material is a nanocomposite material. Preferably, the nanocomposite material is bioactive. Advantageously, the nanocomposite material combines the bioactivity of bioactive glasses with the toughness of biodegradable polymers.

A nanomaterial is a material having structured components with at least one dimension on the nanoscale (less than 100 nm). In the context of the present invention, a ‘nanocomposite material’ is taken to be a composite material, comprising at least two phases, wherein at least one phase comprises a nanomaterial, the two phases being integrated at the nanoscale.

The organic phase and the inorganic phase are integrated at the nanoscale, with interfacial covalent bonding occurring between the phases. This contrasts to conventional composite materials wherein the inorganic and organic phases are not integrated at the nanoscale, instead comprising distinct particles of inorganic material having macroscale dimensions, dispersed within a polymer network.

In a preferred embodiment, the inorganic phase is non-particulate. Preferably, the inorganic phase comprises inorganic chains having at least one dimension on the nanoscale.

In an alternative preferred embodiment, the inorganic phase comprises particles having an averaged maximum diameter no greater than 200 nm, preferably no greater than 100 nm, more preferably no greater than 50 nm, even more preferably between 20 and 50 nm.

In a preferred embodiment, the porous composite material has an interconnected pore network making it suitable for use as a scaffold for promoting bone growth. Preferably, the porous material comprises macropores having a mean diameter up to 500 μm, preferably between 100 and 500 μm. Preferably, the mean minimum dimension of interconnection between macropores is at least 100 μm.

The polymer present in the composite is enzymatically degradable. Thus, preferably the polymer is not a synthetic polyester. The use of a polymer that degrades by cellular and enzymatic mechanisms, rather than purely by hydrolysis, enables the provision of a scaffold that will degrade with a controlled rate from the outside in when implanted in the body. This is in contract to the unpredictable and non-linear degradation rate seen for polymers that degrade solely by hydrolysis, such as polyesters. The polymer may degrade by both enzymatic mechanisms and hydrolysis.

In a preferred embodiment, the polymer has an anionic charge at physiological pH. The anionic charge can be beneficially used to carry metal cations, such as Ca²⁺ ions, into a bone regeneration site.

In a preferred embodiment, the composite material comprises calcium ions coordinated to anionic charges present on the organic polymer and/or integrated within the silica network of the inorganic phase.

In a preferred embodiment, the composite material additionally comprises strontium ions coordinated to anionic charges present on the organic polymer and/or integrated within the silica network of the inorganic phase. Alternatively, strontium ions are present and calcium ions are absent. Strontium ions are useful for promoting bone regeneration.

In a preferred embodiment, the composite material additionally comprises a source of metal ions useful for promoting wound healing and/or revascularisation, for example lithium, copper or cobalt ions.

In a preferred embodiment, the organic polymer comprises functional groups capable of functionalisation to allow covalent bond formation with the inorganic phase. Preferably, the functional groups are capable of silanation. Preferably, the functional groups are hydroxyl and/or carboxyl groups. Silanation is preferably achieved by reaction of the functional groups with a silane crosslinker containing an epoxy functional group, such as glycidoxypropyl trimethoxysilane (GPTMS). Thus, in a preferred embodiment the organic phase is formed from a polymer having pendant hydroxyl and/or carboxyl groups, the inorganic phase comprises a silica network and the organic and inorganic phases are joined by a silane crosslinker containing an epoxy functional group, wherein covalent bonding is present between the crosslinker and both the organic and inorganic phases.

With the use of a silane crosslinker containing an epoxy functional group, covalent bonds are formed between the silane portion of the crosslinker and the inorganic silica network as well as between the epoxy group and the hydroxyl and/or carboxyl groups of the polymer.

Advantageously, the use of a crosslinker enables control of the mechanical properties (e.g. toughness) of the composite material as well as the swelling and degradation rates of the composite material when immersed in an aqueous solution. Too little crosslinking makes the material very flexible as the polymer chains have freedom to move, but allows high water uptake in the material, swelling and high rates of resorption, whereas too much crosslinking will make the nanocomposite brittle due to lack of flexibility of the chains. A balance is needed to obtain a nanocomposite with the desired mechanical properties and a controlled degradation. Preferably, the crosslinker:polymer ratio is 1:50 or lower (in terms of the proportion of crosslinker). The ratio is expressed in terms of the number of monomer units of polymer per crosslinker molecule.

In a preferred embodiment, the molecular weight of the organic polymer is greater than 16000. Preferably, the molecular weight is at least 100000. With a molecular weight of this magnitude good toughness is provided through chain entanglement.

In a preferred embodiment, the composite material comprises from 20 wt % to 70 wt % organic phase. Preferably, the composite material comprises from 20 wt % to 60 wt % organic phase, even more preferably, from 30 wt % to 50 wt %, most preferably 40 wt %. The preferred organic phase proportion is tailored to provide the composite with the desired mechanical properties, i.e. high compressive strength with some toughness

In a preferred embodiment, the polymer is a natural or synthetic polymer. The polymer may be a natural or synthetic polymer that has been derivatised to bear hydroxyl and/or carboxyl functional groups.

In a preferred embodiment, the polymer is a poly-lactide bearing hydroxyl groups, collagen or a derivative thereof such as gelatin, poly (DL aspartic acid) or polyglutamic acid. Preferably, the polymer is poly-α-glutamic acid or poly-γ-glutamic acid. More preferably, the polymer is poly-γ-glutamic acid. More preferably, the polymer is poly-γ-glutamic acid having a poly acrylic acid equivalent molecular weight of 160000 or greater.

Poly-γ-glutamic acid (γ-PGA) is a polymer formed from the monomer glutamic acid and having the following chemical structure:

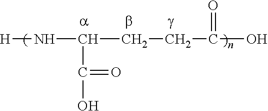

Glutamic acid has three functional groups; α-NH₂, α-COOH and γ-COOH. γ-PGA is a γ-COOH and α-NH₂ peptide linked amino acid. γ-PGA is a natural polymer found in the extracellular matrix. Glutamic acid rich sequences are found in bone at the end of collagen fibrils, where the carboxylic groups are thought to provide nucleation sites for the mineral phase of the bone (Hunter G, The Biochemical Journal, 1996, 302, 175-179). γ-PGA is synthesised by several bacteria, belonging to the Bacillus group. It is produced in several forms: D-, L- or a co-polymer of D and L. Large molecular weights M_{w} in excess of 1.2×10⁶ can be produced with high yield. In the context of the present invention, γ-PGA may be any of D-γ-PGA, L-γ-PGA, a co-polymer of D and L, or any mixture of these forms.

Advantageously, γ-PGA has anionic charge at physiological pH. The anionic charge on the polymer attracts positively charged cations to it. This property can be beneficially used to carry ions such as Ca²⁺ into a regeneration site in which a scaffold comprising the composite material of the invention is implanted. This is a route that allows the safe incorporation of calcium ions into an inorganic/organic hybrid safely. Moreover, the carboxylic acid functional groups (α-COOH) allow silanation of the polymer so that it can be incorporated into the silica network by covalent bonding.

The polymer is functionalised with GPTMS such that the glycidol groups of the GPTMS molecule attach to the carboxylic acid groups on the polymer chain, leaving the three methoxysilane groups free. When the functionalised polymer is added into the sol, the methoxysilane groups hydrolyse, leaving Si—OH groups on the polymer. These groups can then undergo polycondensation with other Si—OH groups in the inorganic network, to form covalent Si—O—Si bonds between the polymer chains and the inorganic network.

For a ratio GPTMS:polymer ratio of 1:50 or below the composite becomes both flexible and tough. The ratio is expressed in terms of the number of monomer units of polymer per GPTMS molecule. Thus, preferably, GPTMS is present at a GPTMS:polymer ratio of 1:50 or lower.

The use of GPTMS not only creates covalent bonds between the inorganic and organic chains, but also allows more polymer to be incorporated into the sol-gel process. The presence of Si—CH₃O groups on the polymer allows incorporation of the polymer during condensation. This reduces phase separation.

Advantageously, γ-PGA is safe and inexpensive (it is known for use as a food additive), it has soluble forms and it can degrade by both hydrolysis and enzymatic degradation. Enzymes responsible for degradation of γ-PGA include γ-glutamyl transpeptidase.

In order to produce a porous composite material according to the first aspect of the invention, a composite is subjected to a foaming process in order to introduce porosity. It will be appreciated that a non-porous composite material can be produced using the same components as set out in the first aspect of the invention, but without subjection to a foaming process. It will be appreciated, therefore, that in a second aspect, the present invention provides a composite material having preferred features as set out for the first aspect, but absent a macroporous structure.

In a third aspect, the present invention provides a process for producing a porous composite material as defined in the first aspect of the invention comprising:
a) silanating an organic polymer;
b) providing an aqueous sol comprising a source of silica, preferably a silica alkoxide;
c) adding the silanated polymer to the sol;
d) adding a surfactant and a gelation catalyst to the sol;
e) agitating the sol in the presence of air to generate a foam; and
f) aging and drying the foam to provide a porous composite material,

wherein a source of calcium and/or strontium ions is incorporated into the composite material by introducing a source of calcium and/or strontium ions into the sol and/or by exposing the porous composite material generated in step e) to an aqueous solution containing calcium and/or strontium ions, preferably after aging and drying.

Preferably, the composite material is a nanocomposite material.

Preferably, the organic polymer is an enzymatically biodegradeable polymer which comprises pendant hydroxyl and/or carboxyl groups. The polymer may be a natural polymer or a synthetic polymer that has been derivatised to bear hydroxyl and/or carboxyl groups. Preferably, the polymer is silanated by reaction of the pendant functional groups (preferably hydroxyl and/or carboxyl groups) with an epoxy-containing silane crosslinker such as glycidoxypropyl trimethoxysilane (GPTMS). Preferably, this reaction is carried out in the presence of a solvent, such as DMSO or water. Preferably, at least a portion of the solvent is removed by evaporation from the resulting silanated-polymer containing mixture prior to addition of the silanated polymer to the sol.

Preferably, the aqueous sol is prepared by reacting a silica alkoxide, preferably tetraethyl orthosilicate (TEOS), with water under acidic catalysis.

Preferably, the source of calcium introduced into the sol is calcium chloride.

Preferably, the gelation catalyst is hydrofluoric acid (preferably provided as an aqueous HF solution).

Preferably, the porous composite material generated in step e) is exposed to an ion rich solution produced by dissolving powdered silica-calcium glass in water. Preferably, the ion rich solution is pumped through the porous material.

Preferably, in step f) the foam is aged at 50-70° C. (preferably 60° C.) and dried at 50-70° C. (preferably 60° C.), under vacuum. Preferably, the step of aging comprises heating to 50-70° C. (preferably 60° C.) for a first period of time (preferably 50-80 hours), cooling and reheating to 50-70° C. (preferably 60° C.) for a second period of time (preferably 80-120 hours).

Therefore, in a fourth aspect, the present invention provides a process for incorporating calcium ions into a porous composite material comprising integrated organic and inorganic phases, wherein the organic phase comprises an enzymatically biodegradable organic polymer and the inorganic phase comprises a sol-gel derived silica network, wherein covalent bonding is present between the organic phase and the inorganic phase, the process comprising exposing the porous material to an ion rich solution produced by dissolving powdered silica-calcium glass in water, by pumping the ion rich solution through the porous material.

It will be appreciated that the preferred features set out in respect of the composite material of the first aspect of the invention apply equally to the composite material produced by the processes of the third and fourth aspects of the invention.

In a fifth aspect, the present invention provides a composite material as defined above for use in medicine. Preferably, the composite material is for use as a scaffold for aiding bone repair and/or regeneration.

In a sixth aspect, the present invention provides a scaffold for bone repair and/or regeneration comprising a composite material as defined in the first aspect of the invention.

All preferred features of each of the aspects of the invention apply to all other aspects mutatis mutandis.

The invention may be put into practice in various ways and specific embodiment will be described to illustrate the invention, with reference to the accompanying figures in which:

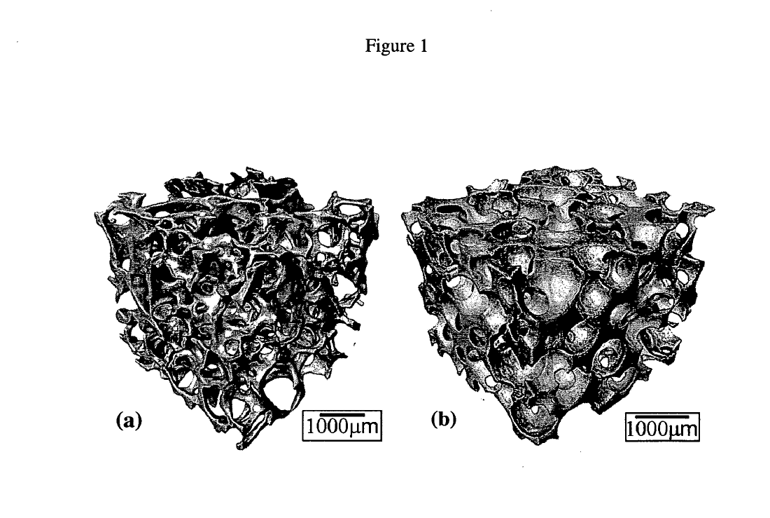

FIG. 1 shows three dimensional (3D) X-ray micro computer tomography (μCT) images of human trabecular bone (FIG. 1a) and a typical bioactive glass scaffold produced by the sol-gel foaming process (FIG. 1b) and shows that the pore network of the scaffolds are very highly interconnected and similar to the pore structure of trabecular bone.

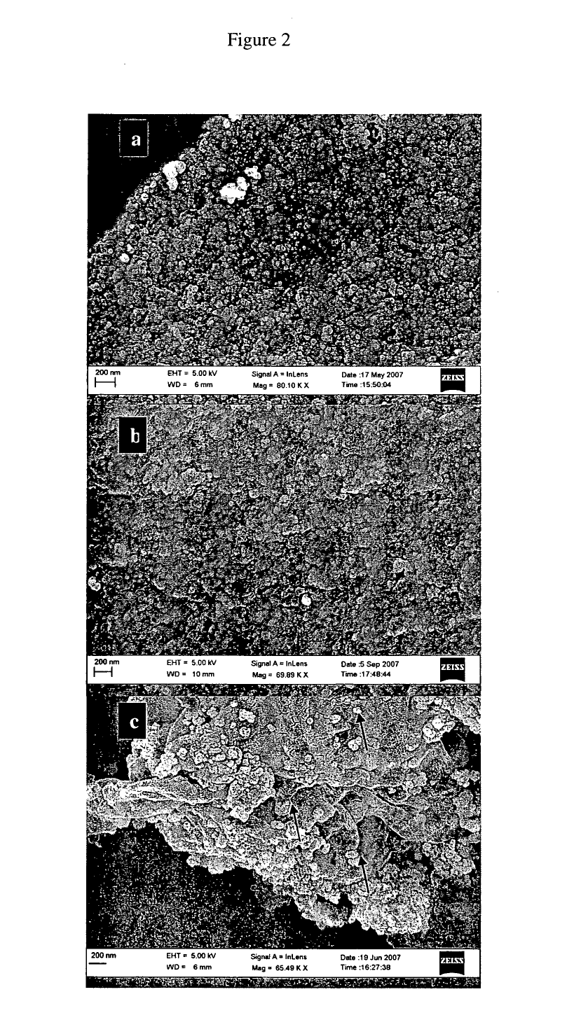

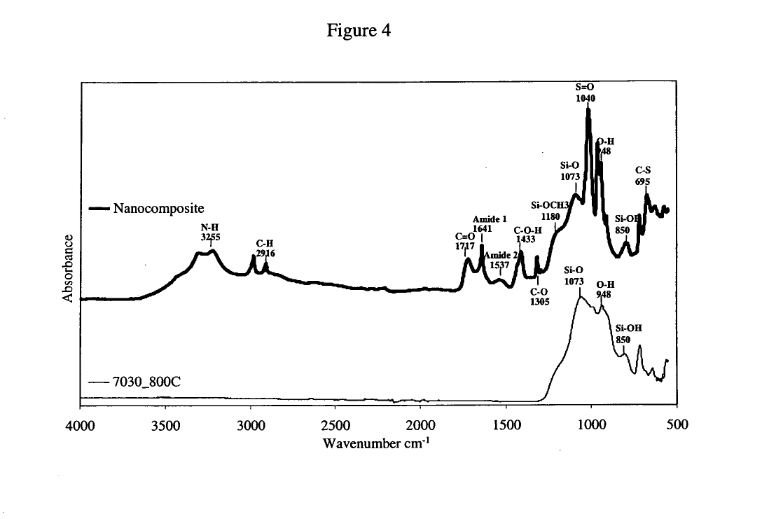

FIG. 2 shows three scanning electron microscopy (SEM) images of three different compositions of the nanocomposite material of the present invention. FIG. 4a) 80 wt % SiO₂ and 20 wt % polymer, 4b) 50 wt % SiO₂ and 50 wt % polymer, and 4c) 30 wt % SiO₂ and 70 wt % polymer. Arrows in 4c point to the attached nanoparticles of SiO₂ at high weight % polymer.

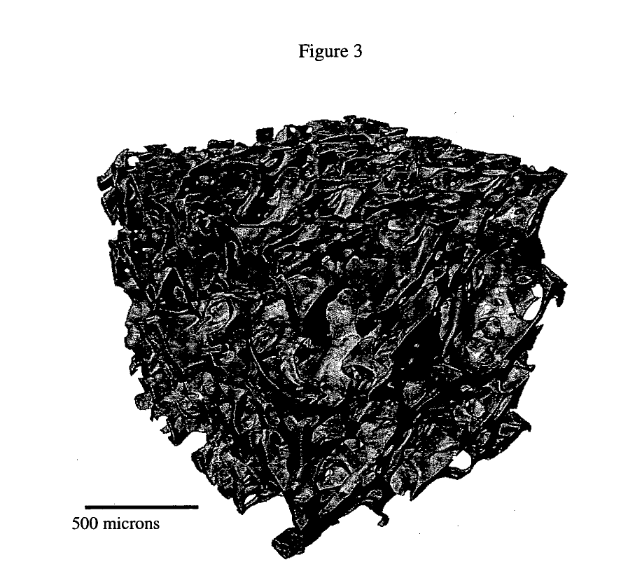

FIG. 3 shows a three dimensional micro computed topography (μCT) image of a nanocomposite material of the present invention.

FIG. 4 shows a FTIR spectra of a 70S30C sol-gel derived bioactive glass and a nanocomposite (containing 40 wt % γ-PGA, with a crosslinker ratio of 1:50). The spectrum for 70S30C shows absorbance bands corresponding to Si—O bonds. The spectrum for the nanocomposite shows that it contains Si—O bonds, some DMSO. Importantly the spectrum also contains bands corresponding to N—H, C—H, C═O, amide I, amide II and C—O—H, indicating that the nanocomposite contains a polymer containing peptide bonds and carboxylic acid groups. This FTIR therefore confirmed presence of a polymer within the nanocomposite.

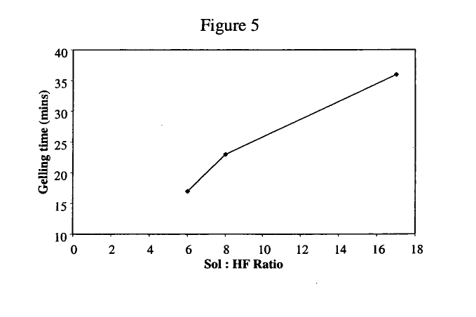

FIG. 5 shows a graph of gelling time as a function of HF content for nanocomposites with a crosslinker:polymer ratio (moles of GPTMS:polymer monomer units) of 1:50 and with 40 vol % of DMSO removed.

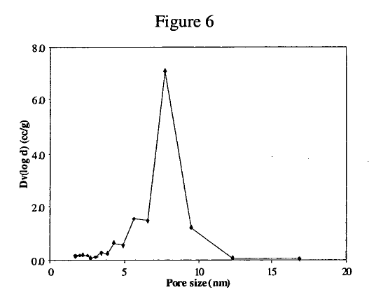

FIG. 6 shows pore size distributions of a nanocomposite, with a crosslinker: polymer molar ratio of 1:50, after it was immersed in water solution for 24 hours.

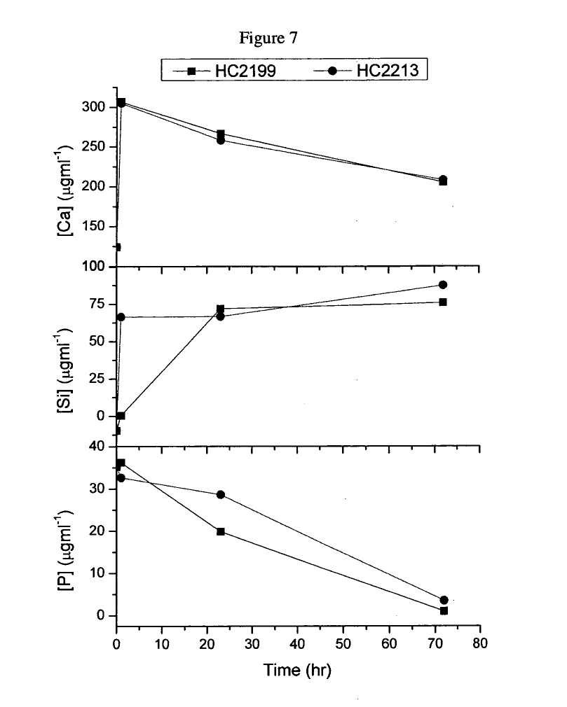

FIG. 7 shows ion release profiles of SBF after immersion of a nanocomposite with 40 wt % γ-PGA and a crosslinker:polymer ratio of 1:50.

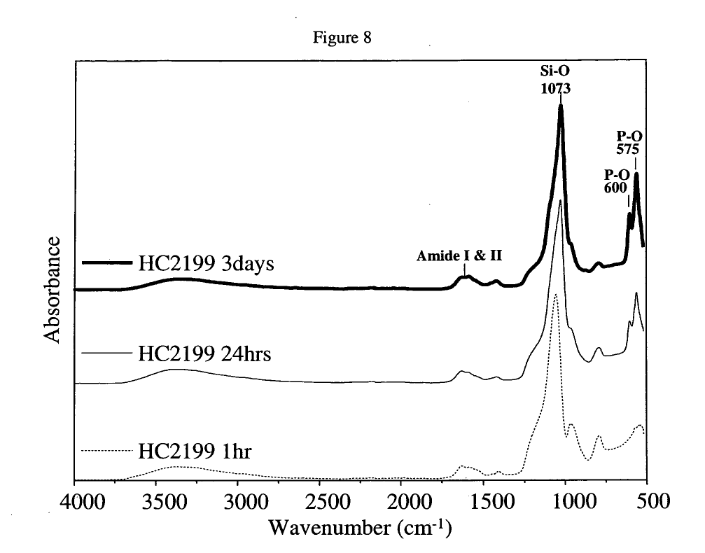

FIG. 8 shows FTIR spectra of a nanocomposite, with 40 wt % γ-PGA and a crosslinker:polymer molar ration of 1:50, after immersion in SBF.

In the context of this invention, a biologically active (or bioactive) material is one which, when implanted into living tissue, induces formation of an interfacial bond between the material and the surrounding tissue. More specifically, bioactive materials induce biological activity that results in the formation of a strong bond between the bioactive material and living tissue such as bone.

Bioactivity is the result of a series of complex physiochemical reactions on the surface of a material under physiological conditions, leading to formation of a hydroxycarbonated apatite (HCA) layer of the surface of the material. The HCA layer that forms is structurally and chemically equivalent to the mineral phase of bone and allows the creation of an interfacial bond between the surface of the bioactive material and living tissue.

The rate of development of the hydroxycarbonated apatite (HCA) layer provides an in vitro index of bioactivity. Bioactivity can be effectively examined by using non-biological solutions that mimic the fluid compositions found in relevant implantation sites within the body. Investigations have been performed using a variety of these solutions including Simulated Body Fluid (SBF), as described in Kokubo T, J. Biomed. Mater. Res. 1990; 24; 721-735. Deposition of an HCA layer on a material exposed to SBF is a recognised test of bioactivity and, in the context of the present invention, a material is considered to be bioactive if, on exposure to SBF, deposition of a crystalline HCA layer occurs within three days. In some preferred embodiments, HCA deposition occurs within 24 hours.

In addition, the surface of a material exposed to SBF can be monitored for the formation of an HCA layer by X-ray powder diffraction and Fourier Transform Infra Red Spectroscopy (FTIR). The appearance of hydroxycarbonated apatite peaks, characteristically at two theta values of 25.9, 32.0, 32.3, 33.2, 39.4 and 46.9 in an X-ray diffraction pattern is indicative of formation of a HCA layer, as is the appearance of a P—O bend signal at a wavelength of 566 and 598 cm⁻¹ in an FTIR spectra is indicative of deposition of an HCA layer.

A schematic view of the synthesis of a nanocomposite material of the invention, excluding the step of incorporation of Ca²⁺ ions is set out below:

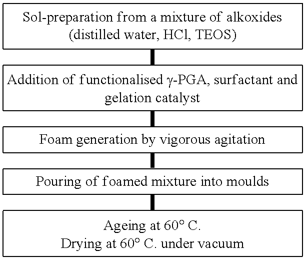

A source of Ca²⁺ or Sr²⁺ ions is incorporated into the nanocomposite by inclusion within the sol or by exposure of the foamed material to a solution containing Ca²⁺ or Sr²⁺ ions, preferably after aging and drying. A detailed description of the synthesis of a nanocomposite material of the invention is set out in the following examples.

Nanocomposite materials have been synthesised and their structure analysed. As shown by the high resolution Scanning electron microscopy (SEM) images set out in FIG. 2, the nano-structure of a nanocomposite material can be tailored depending on the relative amounts of organic and inorganic phase present and the type of gelation catalyst (gelling agent) used in preparation of the nanocomposite material. In certain embodiments, hydrofluoric acid is used as a gelling agent. HF accelerates the hydrolysis and polycondensation of the inorganic silicate. Generally, when the polymer is added to the sol it is already fairly crosslinked to itself. Once in the sol it will undergo cross-linking with silica to give interlinked polymer and inorganic networks. Depending on the relative proportions of the inorganic and organic phases, these networks may take the form of interlinked polymer chains and inorganic (silica) chains. Where the inorganic phase has high wt % (for example in the region of 50 wt % to 80 wt %, the inorganic phase comprises an interlinked silica matrix with polymer chains dispersed therein. As the proportion of the organic phase is increased, for example to a high wt % polymer of the order of 70 wt %, a polymer is the matrix phase is observed with nanoparticles of silica bonded to it. Without being bound to theory, it is thought that this kind of morphology is only possible if the gelling agent used is one that gels the polymer. A close look at the nano-structure of bone reveals that it is composed of straight collagen molecules with apatite mineral crystals at the ends and gaps of the collagen molecules. There are strong bonds present between molecules within each phase and to the other phases at this nanoscale. Consequently, the ideal bonding scenario for a nanocomposite material is one where both the organic polymer phase and the inorganic gel together to form a matrix together, with there being no distinctions between the organic polymeric and inorganic phases.

As well as showing bioactivity and good mechanical properties, the composite materials of the present invention show improved degradation characteristics, particularly in contrast to composite materials containing polymers that degrade solely by hydrolysis, such as polyesters. When polyesters degrade, it is by chain scission due to hydrolysis. Once water uptake has occurred, the polymer chains are cut repeatedly, at the ester bond due to reaction with water, reducing the molecular weight of the polymer. No degradation is observed until the molecular weight drops below the entanglement value for the polymer. Below this value, the chains unravel and the polymer will disintegrate. This is an auto-catalytic process. Any degradation of the polyester results in release of carboxylic acid and a drop in local pH, which will accelerate degradation. The autocatalysis can also cause degradation to occur inside a polyester material. Therefore polyesters may degrade more rapidly in their centre than at the edge, which leads to rapid loss of strength before any loss of mass. In contrast enzymatic degradation would result in degradation from the surface inwards only, allowing bone to replace the scaffold structure progressively.

The invention is further illustrated by reference to the following non-limiting examples:

### EXAMPLES

Preparation of a Nanocomposite Material

Polymer Functionalisation

The first step carried out was silanation of γ-PGA, having a molecular weight of approximately 140000, by reaction with glycidoxypropyl trimethoxysilane (GPTMS).

5 g γ-PGA was placed in a 100 ml capacity 3-necked round bottom flask, to which 45 ml of dimethyl sulfoxide (DMSO) was added as a solvent. A condenser was placed on the centre neck of the flask and two stoppers placed in the side necks. The mixture was heated to 70° C. in an oil bath while mixing with magnetic stirrer. Once the polymer was fully dissolved the temperature was increased to 80° C. and a dry nitrogen flow at a constant speed was attached to one of the side necks of the flask.

In a separate glass container 1.72 ml of (98%) glycidoxypropyl trimethoxysilane (GPTMS) was mixed with 5 ml of DMSO. This GPTMS+DMSO mixture was then added drop-wise to the γ-PGA/DMSO solution. The mixture was allowed to react for 8 hours under the dry N₂.

The crosslinker:polymer ratio in the preparation described above is 1:50.

Preparation of the Sol Mixture

The sol was prepared by reacting tetraethyl orthosilicate (TEOS) with water under acidic catalysis. 19.5 ml deionised water was mixed with 7.8 ml of 1N hydrochloric acid with a magnetic stirrer at room temperature. After five minutes, 2 ml of TEOS was added slowly and allowed to mix for 1 hour. This produced the 100S sol.

To produce a calcium containing sol, a proportional amount of CaCl₂ was added to the 100S sol, and allowed to mix for a further hour. It should be noted that some amount of chlorine can be tolerated by the body, as chlorine is present in the physiological fluid.

Hybrid Synthesis

A water bath was pre-heated to 80° C. The hot functionalised polymer mixture was poured into a 500 ml single necked round bottomed flask. The flask was attached to a rotary vacuum evaporator (RVE) and immersed into the water bath. The rotation speed was set to high for the first 30 minutes and then reduced to very slow for the remaining 30 minutes. A high vacuum is required to evaporate DMSO.

Once more than 40 ml of DMSO was evaporated the RVE was stopped. The 100 S or calcium containing sol mixture was then poured into the silanated polymer and allowed to mix at room temperature with a magnetic stirrer for a day.

Foaming

10 ml aliquots of sol were decanted into a polypropylene beaker, to which 0.6 ml of 5 vol % HF (catalyst solution, 5 vol % or 4.4 wt % in water) and 0.05 ml of surfactant (Teepol, Thames Mead Ltd.) were added. The solution was foamed with vigorous agitation in air. 5 ml of water was added after 5 minutes of mixing to improve the efficiency of the surfactant. Just before gelling pour foam into glass or poly tetrafluoroethylene (PTFE) moulds, which were immediately sealed.

Heat Treatment

The sealed moulds were transferred to a programmable oven and heated to 60° C. at 0.5° C./min for 72 hours, then allowed to cool. The caps were then unscrewed to allow vapour release during drying. The samples were then re-heated to 60° C. for another 100 hours and allowed to cool. The samples were then dried in the vacuum oven in the fume cupboard and heated to 60° C.

Incorporation of Calcium

As an alternative or in addition to incorporating calcium within the sol, calcium ions can be incorporated into the nanocomposite by exposure of the foam produced above to an aqueous solution containing Ca²⁺ ions. This has been achieved by grinding a 70 wt % SiO₂, 30 wt % CaO glass to a powder, dissolving the powder in water to produce an ion rich solution and pumping this solution through the foam to allow coordination of cations with anionic charges present thereon. This pumping method can therefore be used to introduce calcium ions into nanocomposites produced from a 100% silica (100 S) inorganic phase. Good bioactivity as determined by HCA deposition on exposure to SBF has been observed for nanocomposite material produced both by both methods of calcium incorporation.

Effect of Catalyst Concentration on Gelling Time

Increasing the amount of catalyst used in the gelation and foaming step decreases the gelling time, meaning that it is faster to produce the foam scaffolds as less agitation time is needed. However, it is preferable to use low amounts of catalyst because HF can be toxic to human body. In conventional sol-gel glass processing, the HF is removed by heat treatment at around 600-800° C. In contrast, in the process of the invention the HF is removed by low temperature drying and washing, reinforcing the desirability to keep HF content low to reduce the risk of any remaining. A balance between gelling time and HF concentration can be achieved and this is dependent on the volume of DMSO removed prior to the foaming step. For a conventional sol-gel glass, with a sol-gel solution volume to HF (4.4 wt %, solution of HF in water) volume ratio of 50:3 (Sol:HF), gelling takes up to 12 minutes. For nanocomposites with 40 wt % γ-PGA and up to 80 vol % DMSO removed, gelling is complete in 6 minutes for the same Sol:HF ratio. FIG. 5 shows a graph of gelling time as a function of HF content for nanocomposites with a crosslinker:polymer ratio (moles of GPTMS:polymer monomer units) of 1:50 and with 40 vol % of DMSO removed. Gelling time increased as Sol:HF ratio (determined after DMSO removal) increased. Table 1, below, shows the gelling time for different amounts of DMSO removed while keeping the Sol:HF ratio constant.

TABLE 1
Gelling time for the amount of DMSO removed:

| DMSO evaporated | |
|---|---|
| (vol %) | Gelling time (mins) |
| 45 | 14.5 |
| 50 | 17.0 |
| 60 | 3.5 |
| 65 | 3.0 |
| 88 | 3.0 |

Catalyst concentration and gelling time are dependent on the volume % of DMSO evaporated. For example, for 50 vol % DMSO removal, an ideal Sol:HF ratio is 33:1, whereas for 80 vol % DMSO removal, the ideal sol:HF ratio is 17:1.

Imaging of Produced Porous Nanocomposite

Three dimensional micro computed topography (μCT) imaging of a nanocomposite material produced as described above demonstrates that the foaming techniques used is successful in producing a highly porous, well interconnected pore network (see FIG. 3).

In addition, scanning electron microscopy (SEM) was carried out on three different compositions of the nanocomposite material of the present invention and the images generated are shown in FIG. 2, where FIG. 2a) shows a composite with 80 wt % SiO₂ and 20 wt % polymer, FIG. 2b) shows a composite with 50 wt % SiO₂ and 50 wt % polymer, and FIG. 2c) shows a composite with 30 wt % SiO₂ and 70 wt % polymer. For the composite with 70 wt % polymer, silica nanoparticles were observed. Nanoparticles were not observed for the 20 wt % and 50 wt % polymer composites.

Stability Testing

For comparative purposes, stability tests were carried out on an inorganic foam comprising 100% SiO₂ by immersion into simulated body fluid (SBF). The inorganic foam was found to be very stable. For further comparative purposes, hybrids were produced according to the methods set out above, but without silanation of the polymer. The stability of these hybrids was observed to decrease with increasing polymer content. Stability tests carried out on composites produced according to the method set out above demonstrate that this decrease in stability is overcome by silination of the polymer and consequent cross-linking to the silica network. Composites in which silinated polymer was used show an improved modulus and fracture strength.

Extent of Crosslinking

Nanocomposites were prepared as described above with varying crosslinker:polymer ratios. For a high crosslinker: polymer ratio of 1:25 some brittleness is observed, whereas at a ratio of 1:50 or below the nanocomposite becomes both flexible and tough. The ratios are expressed in terms of the number of monomer units of polymer per GPTMS molecule. The desired flexibility and toughness was also observed at a ratio of 1:100.

SBF Bioactivity Testing

Simulated body fluid (SBF) was prepared according to the method of Kokubo, T., et al., J. Biomed. Mater. Res., 1990. 24: p. 721-734. The reagents shown in the table below were added, in order, to deionised water, to make 1 litre of SBF. All the reagents were dissolved in 700 ml of deionised water and warmed to a temperature of 37° C. The pH was measured and HCl was added to give a pH of 7.25 and the volume made up to 1000 ml with deionised water.

TABLE A
Reagents for the preparation of SBF

| Order | Reagents | Amount | |
|---|---|---|---|
| 1 | NaCl | 7.996 | g |
| 2 | NaHCO₃ | 0.350 | g |
| 3 | KCl | 0.224 | g |
| 4 | K₂HPO₄•3H₂O | 0.228 | g |
| 5 | MgCl₂•6H₂O | 0.305 | g |
| 6 | 1N HCL | 35 | ml |
| 7 | CaCl₂•2H₂O | 0.368 | g |
| 8 | Na₂SO4 | 0.071 | g |
| 9 | (CH₂OH)CNH₂ | 6.057 | g |

Nanocomposite materials were exposed to SBF and the deposition of an HCA layer was monitored. SBF bioactivity testing was carried out on a nanocomposite comprising an inorganic phase of 100% SiO₂ (composite 1) and a nanocomposite of the invention produced as described above comprising an inorganic phase of 85% SiO₂ and 15% CaO (composite 2). No hydroxyl carbonate apatite (HCA) layer was observed on composite 1 within 3 days, but an HCA layer was observed on composite 2 within 3 days.

Pore Size Distribution

FIG. 6 shows pore size distributions of a nanocomposite, with a crosslinker:polymer molar ration of 1:50, after it was immersed in water for 24 hours. The modal nanopore size of the nanocomposite was 7.8 nm according to the BJH model (a model used in analysis of nitrogen sorption data that give a pore size distribution). Prior to immersion in SBF, the nanocomposite showed no nanoporosity. The release of un-crosslinked polymer into the water opens up the nanopores, with the silica network remaining intact. Bioactive sol-gel glasses of the 70S30C composition commonly have modal nanopore values of ˜12 nm. The smaller modal nanopore size seen for the composite material of the invention could beneficially attract cell attachment.

ICP Data of Nanocomposite Materials Post Immersion in SBF

ICP indicates migration of Ca & PO₄ to surface to form a CaPO₄ layer. Ion release profiles of SBF after immersion of a nanocomposite with 40 wt % γ-PGA and a crosslinker:polymer molar ration of 1:50 are shown in FIG. 7. The nanocomposite released silicon ions into the SBF as a function of time. In contrast, the Ca and P content in the SBF decreased over time, indicating deposition of a calcium phosphate layer on the surface of the nanocomposite. Calcium phosphate deposition is indicative of the formation of a hydroxycarbonate apatite (HCA) layer, which can form a bond to the apatite in bone, indicating bioactivity.

FIG. 8 shows FTIR spectra of the nanocomposite as processed and then after 1 h, 24 h and 72 h of immersion in SBF. The spectra show that an HCA layer formed within 24 h of immersion in SBF. This is a similar time as it takes an HCA layer to form on a 70S30C bioactive glass.

Preparation of Nanocomposite Materials with Different Compositions

Gelatin Nanocomposite

Gelatin is a natural polymer that has also been used to create a nanocomposite, based on the process as described above for γ-PGA. Tough and flexible scaffolds were produced using GPTMS as a cross-linking agent. A similar method of production was used to that used for the γ-PGA nanocomposites described above. Gelatin was functionalised with GPTMS, using water as a solvent instead of DMSO. Percentages of gelatin used were up to 80 wt %. Flexibility within the nanocomposite material was seen to increase with the percentage of gelatin. The ratio of GPTMS to gelatin was again determined to be important in tailoring the properties of the nanocomposite material. The GPTMS:gelatin molar ratios used were 0, 100, 250, 500, 1000, 1500 and 2000. Phase separation was observed below 500. As GPTMS was increased beyond 1000, unreacted GPTMS was observed in the material. Therefore, the minimum ratio is 100, the maximum is 2000 and the optimum concentration range of GPTMS was 500-1000.

Nanocomposites Produced Using Alternative Crosslinkers

As an alternative to GPTMS, nanocomposite production was attempted using aminopropyltriethoxysilane. The composite material produced using this crosslinker had ionic rather than covalent crosslinking between the organic and inorganic phases. Similar results would be seen for other crosslinkers having organo-functional groups rather than the epoxy group of GPTMS.

It should be understood that the invention is susceptible to various modifications and alternative forms. The invention is not to be limited to the particular forms disclosed, but should cover all modifications, equivalents and alternatives falling within the spirit of the disclosure.

1. A bioactive porous composite material comprising an organic phase and an inorganic phase, wherein the organic and inorganic phases are integrated and wherein the organic phase comprises an enzymatically biodegradable organic polymer and the inorganic phase comprises a sol-gel derived silica network, wherein covalent bonding is present between the organic phase and the inorganic phase and wherein the composite material comprises a source of calcium and/or strontium ions.

2. The material of claim 1, wherein the material is a nanocomposite material.

3. The material of claim 1, wherein the inorganic phase is predominantly non-particulate.

4. The material of claim 1, wherein the inorganic phase comprises inorganic chains having at least one dimension on the nanoscale.

5. The material of claim 1, wherein the inorganic phase comprises particles having an average maximum diameter no greater than 200 nm.

6. The material of claim 1, wherein the material has an interconnected porous network comprising macropores having a mean diameter up to 500 μm.

7. The material of claim 6, wherein the mean minimum dimension of interconnection between macropores is at least 100 μm.

8. The material of claim 1, wherein the polymer has an anionic charge at physiological pH.

9. The material of claim 1, comprising calcium ions coordinated to anionic charges present on the organic polymer and/or integrated within the silica network of the inorganic phase.

10. The material of claim 1, wherein the material comprises strontium ions coordinated to anionic charges present on the organic polymer and/or integrated within the silica network of the inorganic phase.

11. The material of claim 1, wherein the polymer comprises a functional group capable of silanation.

12. The material of claim 11, wherein the polymer comprises hydroxyl and/or carboxyl groups.

13. The material of claim 1, wherein the organic phase is formed from a polymer having pendant hydroxyl and/or carboxyl groups, the inorganic phase comprises a silica network and the organic and inorganic phases are joined by a silane crosslinker containing an epoxy functional group, wherein covalent bonding is present between the crosslinker and both the organic and inorganic phases.

14. The material of claim 1, wherein the molecular weight of the organic polymer is greater than 16000.

15. The material of claim 1, wherein the composite material comprises from 20 wt % to 70 wt % organic phase.

16. The material of claim 1, wherein the polymer is a poly-lactide bearing hydroxyl groups, collagen or a derivative thereof such as gelatin, poly (DL aspartic acid) or polyglutamic acid.

17. The material of claim 16, wherein the polymer is poly-α-glutamic acid or poly-γ-glutamic acid.

18. A bioactive nanocomposite material comprising integrated organic and inorganic phases, wherein the organic phase comprises a biodegradable organic polymer and the inorganic phase comprises a sol-gel derived silica network, wherein covalent bonding is present between the organic phase and the inorganic phase and wherein the nanocomposite material comprises a source of calcium ions.

19. A process for producing a porous composite material as defined in claim 1 comprising:
a) silanating an organic polymer;
b) adding the silanated polymer to the sol an aqueous sol comprising a source of silica;
c) adding a surfactant and a gelation catalyst to the sol;
d) agitating the sol in the presence of air to generate a foam; and
e) aging and drying the foam to provide a porous composite material, wherein calcium is incorporated into the composite material either by introducing a source of calcium and/or strontium ions is incorporated into the sol and/or by exposing the porous composite material generated in step d) to an aqueous solution containing calcium and/or strontium ions.

20. The process of claim 19, wherein the organic polymer is an enzymatically biodegradeable polymer.

21. The process of claim 20, wherein the organic polymer comprises hydroxyl and/or carboxyl functional groups.

22. The process of claim 21, wherein the polymer is silanated by reaction of the pendant functional groups with an epoxy-containing silane crosslinker.

23. The process of claim 19, wherein the aqueous sol is prepared by reacting a silica alkoxide with water under acidic catalysis.

24. The process of claim 19, wherein the source of calcium introduced into the sol is calcium chloride and/or wherein the gelation catalyst is hydrofluoric acid.

25. The process of claim 19, wherein the porous nanocomposite material generated in step d) is exposed to an ion rich solution produced by dissolving powdered silica-calcium glass in water, by pumping the ion rich solution through the porous material.

26. A process for incorporating calcium ions into a porous nanocomposite material comprising integrated organic and inorganic phases, wherein the organic phase comprises an enzymatically biodegradable organic polymer and the inorganic phase comprises a sol-gel derived silica network, wherein covalent bonding is present between the organic phase and the inorganic phase, the process comprising exposing the porous material to an ion rich solution produced by dissolving powdered silica-calcium glass in water, by pumping the ion rich solution through the porous material.

27. (canceled)

28. The composite material of claim 27, for use as a scaffold A method for aiding bone repair and/or regeneration in a human comprising administering a composite material of claim 1 to the human.

29-30. (canceled)
